(19) United States
(12) Patent Application Publication
(10) Pub. No.: US 2014/0100184 A1
(43) Pub. Date: Apr. 10, 2014

(54) SELECTIVE INHIBITORS OF HISTONE METHYLTRANSFERASE DOT1L

(71) Applicant: BAYLOR COLLEGE OF MEDICINE, Houston, TX (US)

(72) Inventors: Yongcheng SONG, Pearland, TX (US); Pinhong CHEN, Houston, TX (US); Jiasheng DIAO, Houston, TX (US); Gang CHENG, Houston, TX (US); Lisheng DENG, Houston, TX (US); Justin L. ANGLIN, Houston, TX (US); Venkataram B.V. PRASAD, Houston, TX (US); Yang YAO, Houston, TX (US)

(73) Assignee: BAYLOR COLLEGE OF MEDICINE, Houston, TX (US)

(21) Appl. No.: 14/015,913

(22) Filed: Aug. 30, 2013

Related U.S. Application Data
(60) Provisional application No. 61/695,720, filed on Aug. 31, 2012.

(51) Int. Cl. C07H 19/16; C12Q 1/48

(52) U.S. Cl. CPC C07H 19/16; C12Q 1/48
USPC 514/46; 536/27.23; 435/15

(57) ABSTRACT

Structure and mechanism based design was used to design potent ribose containing inhibitors of DOT1L with IC₅₀ values as low as 38 nM. These ribose containing inhibitors exhibit only weak or no activities against four other representative histone lysine and arginine methyltransferases, G9a, SUV39H1, PRMT1 and CARM1.

### CROSS-REFERENCE TO RELATED APPLICATIONS

This application claims priority to U.S. Provisional Application No. 61/695,720, filed Aug. 31, 2012.

### BACKGROUND

1. Field of the Invention

The invention relates generally to the design and synthesis of ribose and non-ribose containing selective inhibitors of histone methyltransferase DOT1 L.

2. Background of the Invention

Histone H3-lysine79 (H3K79) methyltransferase DOT1 L plays critical roles in normal cell differentiation as well as initiation of acute leukemia. Thus potent inhibitors of DOT1 L with low IC₅₀ values that are highly selective, and do not inhibit other representative histone lysine and arginine methyltransferases, such as G9a, SUV39H1, PRMT1 and CARM1 are particularly desirable to target acute leukemia. These and other such needs are addressed by embodiments of the disclosure described herein.

### BRIEF SUMMARY OF THE DISCLOSED EMBODIMENTS

These and other needs in the art are addressed in one embodiment of the present invention by a compound of formula 1:

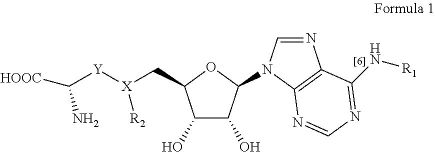

or a pharmaceutically acceptable salt or prodrug thereof, wherein R₁ is H, methyl, or benzyl; R₂ is 2-cyanoethyl, 2-methoxycarbonylethyl, 2-iodoethyl; X is N or S; wherein if X═S, R₂=0; and Y is C3 or C4, wherein said compound is selective for DOT1 L Methyl Transferase.

In a further embodiment, a compound of Formula 2:

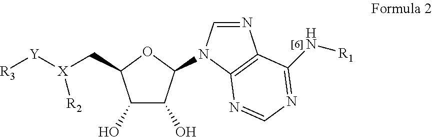

or a pharmaceutically acceptable salt or prodrug thereof, wherein R₁ is H; alkyl; or benzyl; R₂ is H, 2-cyanoethyl, 2-methoxycarbonylethyl, methyl, 2-iodoethyl; ethanol; butyl; benzyl carbamate; X is N; C; or S; wherein if X═S, R₂=0; and wherein if X═C, R₂ is also equal to R₃ or R₁, and Y is also equal to R₁, R₂ or R₃; Y is C, C₂,C₃ or C₄; R₃ is H or selected from the following:

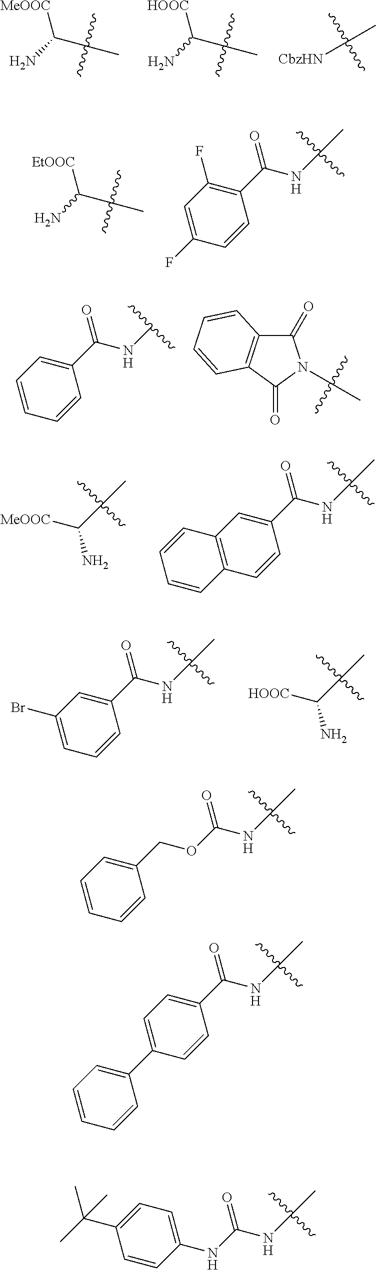

In another embodiment, a compound of Formula 3:

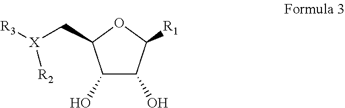

or a pharmaceutically acceptable salt or prodrug thereof, wherein R₁ is H, or a substituted or nonsubstituted: alkyl, cycloalkyl, morpholino, aryl, biaryl, fused biaryl, benzyl; heterocycle, purine, pyrimidine, alcohol, amine, amide, aldehyde, ketone, thiol; ester, ethers, carboxylate, acyl halide, imide, amidine, nitrile, cyano, thioaldehyde, ketone, thione, thioester, thioether, hydrazines, or disulphide; X is C, N, O or S; wherein if X═O, R₂=0; and R₃ is H, O, or R₁; R₂ is H, O, or R₁; or R₃ and R₂ are cyclized together to form a substituted or nonsubstituted: alkyl, cycloalkyl, aryl, biaryl, fused biaryl, benzyl; heterocycle, purine, pyrimidine; and wherein said substituent may be selected from R₁, R₂, R₃, X,halide; or combinations thereof; wherein said compound is selective for DOT1 L Methyl Transferase.

In another embodiment of a compound of claim 1, R1 specifically binds in the hydrophobic pocket comprising Phe 223, Leu224, Val249, Lys187 and Pro133 of DOT1 L protein, thereby selectively inhibiting DOT1 L Methyl Transferase activity. In another embodiment of a compound of claim 2, R1 specifically binds in the hydrophobic pocket comprising Phe 223, Leu224, Val249, Lys187 and Pro133 of DOT1 L protein, thereby selectively inhibiting DOT1 L Methyl Transferase activity. In another embodiment of a compound of claim 3, R1 specifically binds in the hydrophobic pocket comprising Phe 223, Leu224, Val249, Lys187 and Pro133 of DOT1 L protein, thereby selectively inhibiting DOT1 L Methyl Transferase activity.

In one embodiment of a compound of claim 1, the N6 hydrogen forms a hydrogen bond with Asp222 of the DOT1 L protein; thereby selectively inhibiting DOT1 L Methyl Transferase activity. In one embodiment of a compound of claim 2, the N6 hydrogen forms a hydrogen bond with Asp222 of the DOT1 L protein; thereby selectively inhibiting DOT1L Methyl Transferase activity. In one embodiment of a compound of claim 3, the N6 hydrogen forms a hydrogen bond with Asp222 of the DOT1 L protein; thereby selectively inhibiting DOT1 L Methyl Transferase activity.

In an embodiment of a compound of claim 1, the compound has specificity for DOT1L and is substantially free of specificity for CARM1, PRMT1, G9a and SUV39H1 Methyl Transferases.

In an embodiment of a compound of claim 2, the compound has specificity for DOT1L and is substantially free of specificity for CARM1, PRMT1, G9a and SUV39H1 Methyl Transferases.

In an embodiment of a compound of claim 3, the compound has specificity for DOT1L and is substantially free of specificity for CARM1, PRMT1, G9a and SUV39H1 Methyl Transferases.

In another embodiment herein described a composition is provided for, wherein the composition comprises a compound of claim 1 or a pharmaceutically acceptable salt thereof, and a pharmaceutically acceptable carrier. In another embodiment herein described a composition is provided for, wherein the composition comprises a compound of claim 2 or a pharmaceutically acceptable salt thereof, and a pharmaceutically acceptable carrier. In another embodiment herein described a composition is provided for, wherein the composition comprises a compound of claim 3 or a pharmaceutically acceptable salt thereof, and a pharmaceutically acceptable carrier.

One embodiment of the disclosure herein described, provides for a method of treating mixed lineage leukemia in a subject, comprising administering to the subject a therapeutically effective amount of a compound of Formula 1:

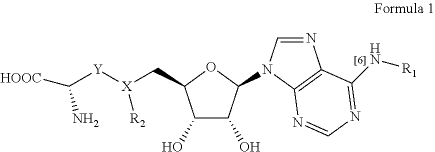

or a pharmaceutically acceptable salt or prodrug thereof, wherein R₁ is H, methyl, or benzyl; R₂ is 2-cyanoethyl, 2-methoxycarbonylethyl, 2-iodoethyl; X is N or S; wherein if X═S, R₂=0; and Y is C3 or C4, wherein said compound is selective for DOT1 L Methyl Transferase.

In an embodiment of a compound of claim 1, the compound has specificity for DOT1L and is substantially free of specificity for CARM1, PRMT1, G9a and SUV39H1 Methyl Transferases, and wherein said compound may be administered as a prodrug; wherein said prodrug comprises replacing RCOOH or RCONH2 with an analogous alkyl ester, an aryl ester, or a heteroaryl ester.

In an embodiment of a compound of claim 2, the compound has specificity for DOT1L and is substantially free of specificity for CARM1, PRMT1, G9a and SUV39H1 Methyl Transferases, and wherein said compound may be administered as a prodrug; wherein said prodrug comprises replacing RCOOH or RCONH2 with an analogous alkyl ester, an aryl ester, or a heteroaryl ester.

In an embodiment of a compound of claim 3, the compound has specificity for DOT1L and is substantially free of specificity for CARM1, PRMT1, G9a and SUV39H1 Methyl Transferases, and wherein said compound may be administered as a prodrug; wherein said prodrug comprises replacing RCOOH or RCONH2 with an analogous alkyl ester, an aryl ester, or a heteroaryl ester.

In another embodiment of the disclosure herein provided is a method of detecting a mixed lineage leukemia comprising, adding a diagnostically effective amount of a compound of formula 1, wherein R₁ is H, methyl, or benzyl; R₂ is 2-cyanoethyl, 2-methoxycarbonylethyl, 2-iodoethyl; X is N or S; wherein if X═S, R₂=0; and Y is C3 or C4, wherein said compound is selective for DOT1 L Methyl Transferase; a pharmaceutically acceptable salt, or prodrug thereof, to an in vitro biological sample.

In a further embodiment of the disclosure herein provided is a method of detecting a mixed lineage leukemia comprising, adding a diagnostically effective amount of a compound of formula 2, wherein R₁ is H; alkyl; or benzyl; R₂ is H, 2-cyanoethyl, 2-methoxycarbonylethyl, methyl, 2-iodoethyl; ethanol; butyl; benzyl carbamate; X is N; C; or S; wherein if X═S, R₂=0; and wherein if X═C, R₂ is also equal to R₃ or R₁, and Y is also equal to R₁, R₂ or R₃; Y is C, C₂,C₃ or C₄; R₃ is H or selected from the following:

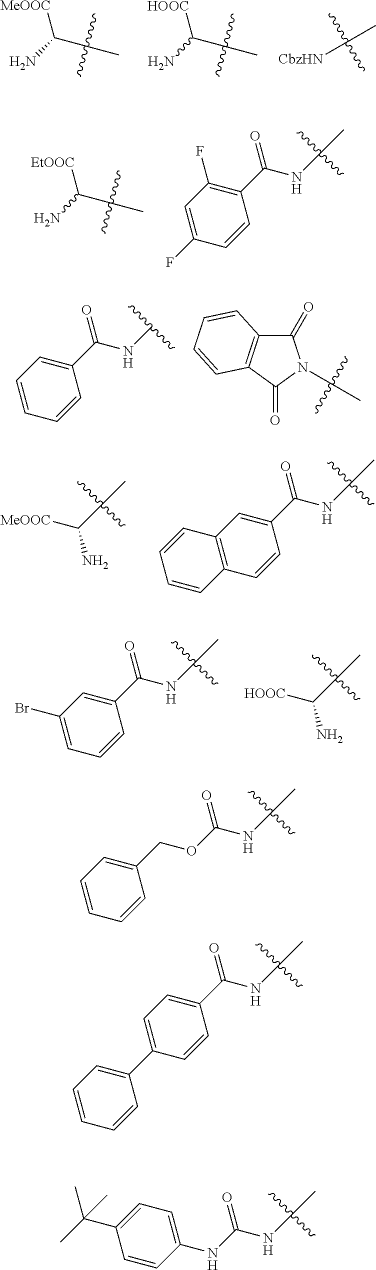

a pharmaceutically acceptable salt, or prodrug thereof, to an in vitro biological sample.

In a further still embodiment of the disclosure herein provided is a method of detecting a mixed lineage leukemia comprising, adding a diagnostically effective amount of a compound of formula 3, wherein R₁ is H, or a substituted or nonsubstituted: alkyl, cycloalkyl, morpholino, aryl, biaryl, fused biaryl, benzyl; heterocycle, purine, pyrimidine, alcohol, amine, amide, aldehyde, ketone, thiol; ester, ethers, carboxylate, acyl halide, imide, amidine, nitrile, cyano, thioaldehyde, ketone, thione, thioester, thioether, hydrazines, or disulphide; X is C, N, O or S; wherein if X═O, R₂=0; and R₃ is H, O, or R₁; R₂ is H, O, or R₁; or R₃ and R₂ are cyclized together to form a substituted or nonsubstituted: alkyl, cycloalkyl, aryl, biaryl, fused biaryl, benzyl; heterocycle, purine, pyrimidine; and wherein said substituent may be selected from R₁, R₂, R₃, X,halide; or combinations thereof; or a pharmaceutically acceptable salt or prodrug thereof, to an in vitro biological sample.

In another embodiment of the disclosure herein provided is a method of detecting a mixed lineage leukemia comprising, administering to the subject an effective amount of a compound of formula 1, wherein R₁ is H, methyl, or benzyl; R₂ is 2-cyanoethyl, 2-methoxycarbonylethyl, 2-iodoethyl; X is N or S; wherein if X═S, R₂=0; and Y is C3 or C4, wherein said compound is selective for DOT1 L Methyl Transferase; a pharmaceutically acceptable salt, or prodrug thereof; and in a further embodiment the subject is human.

In a further embodiment of the disclosure herein provided is a method of detecting a mixed lineage leukemia comprising, administering to the subject an effective amount of a compound of formula 2, wherein R₁ is H; alkyl; or benzyl; R₂ is H, 2-cyanoethyl, 2-methoxycarbonylethyl, methyl, 2-iodoethyl; ethanol; butyl; benzyl carbamate; X is N; C; or S; wherein if X═S, R₂=0; and wherein if X═C, R₂ is also equal to R₃ or R₁, and Y is also equal to R₁, R₂ or R₃; Y is C, C₂,C₃ or C₄; R₃ is H or selected from the following:

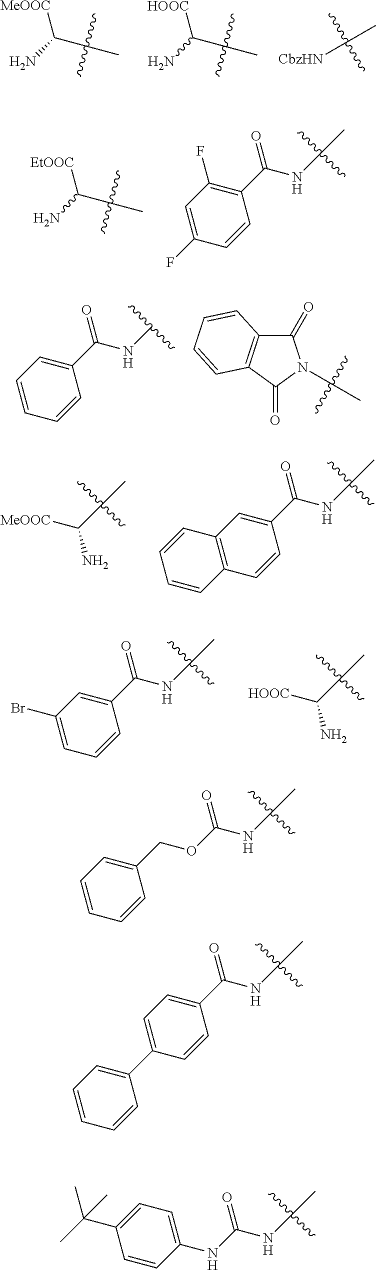

a pharmaceutically acceptable salt, or prodrug thereof; and in a further embodiment the subject is human.

In a further still embodiment of the disclosure herein provided is a method of detecting a mixed lineage leukemia comprising, administering to the subject an effective amount of a compound of formula 3, wherein R₁ is H, or a substituted or nonsubstituted: alkyl, cycloalkyl, morpholino, aryl, biaryl, fused biaryl, benzyl; heterocycle, purine, pyrimidine, alcohol, amine, amide, aldehyde, ketone, thiol; ester, ethers, carboxylate, acyl halide, imide, amidine, nitrile, cyano, thioaldehyde, ketone, thione, thioester, thioether, hydrazines, or disulphide; X is C, N, O or S; wherein if X═O, R₂=0; and R₃ is H, O, or R₁; R₂ is H, O, or R₁; or R₃ and R₂ are cyclized together to form a substituted or nonsubstituted: alkyl, cycloalkyl, aryl, biaryl, fused biaryl, benzyl; heterocycle, purine, pyrimidine; and wherein said substituent may be selected from R₁, R₂, R₃, X,halide; or combinations thereof; or a pharmaceutically acceptable salt or prodrug thereof; and in another embodiment the subject is human.

In a further embodiment, compounds comprising formula 1, formula 2, or formula 3 specifically inhibit methylation of histone3 lysine79 residues located in nucleosome core structure.

In another embodiment, the disclosure herein provides for a method of treating mixed lineage leukemia in a subject, comprising administering to the subject a therapeutically effective amount of compound, wherein said compound is a structural mimic of a reaction intermediate of a compound comprising formula 1, formula 2, or formula 3.

### BRIEF DESCRIPTION OF THE DRAWINGS

For a detailed description of the disclosed embodiments of the invention, reference will now be made to the accompanying drawings, wherein:

FIG. 1 depicts the mechanism of catalysis of DOT1 L;

FIG. 2 (A-C) is an X-ray crystal structure of the human DOT1 L:1 complex (FIG. 2A) is a superposition of the structures of DOT1 L:1 (with C atoms in green) and DOT1 L:SAM (in purple) with a rms deviation of 0.2 Å. For clarity, only protein backbones are shown; (FIG. 2B) Close-up view of the active site of DOT1 L:1 structure, with 10 H-bonds shown in dotted lines; (FIG. 2C) Electrostatic potential surface (with 25% transparency) of the DOT1 L:1 complex, showing the N6-methyl group of 1 is located in a hydrophobic cavity. 1 is shown as a space-filling model;

FIG. 3: Crystal structures of (A) DOT1 L:SAM (PDB: 1NW3); (B) CARM1:SAH (PDB: 2V74); and (C) G9a:SAH (PDB: 3K5K), showing the 6-NH₂ (the two H atoms highlighted in black) of SAM forming only one H-bond with DOT1 L (A, left) with a mainly hydrophobic cavity nearby (A, right). The 6-NH₂ group of SAH forms two H-bonds with CARM1 and G9a (B and C). Electrostatic potential molecular surfaces of the proteins are shown with 20% transparency;

FIG. 4; Mechanism of inhibition of selective inhibitor of an embodiment disclosed herein;

FIGS. 5A and 5B are dose response curves for DOT1 L inhibited by compounds SAH, 1-6, made in accordance with principles described herein;

FIG. 6 (A-D) depicts (A) The overall structure of human DOT1 L in complex with compound 1; (B) Protein-ligand interaction diagram for 1 in DOT1L; (C) The 2Fₒ-F_{c} electron density map of 1, contoured at 1σD. The Fₒ-F_{c} omit map of 1, contoured at 3σ.

FIG. 7 depicts the mechanism of action of compounds 3-6, which were made in accordance with principles described herein; and

FIG. 8: Examples of compounds synthesized by the embodiments methods herein described are illustrated (Syc-377 through SYC-466).

### DETAILED DESCRIPTION OF THE DISCLOSED EMBODIMENTS

The following discussion is directed to various exemplary embodiments of the invention. However, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and that the scope of this disclosure, including the claims, is not limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may be omitted in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct engagement between the two devices, or through an indirect connection via other intermediate devices and connections. As used herein, the term “about,” when used in conjunction with a percentage or other numerical amount, means plus or minus 10% of that percentage or other numerical amount. For example, the term “about 80%,” would encompass 80% plus or minus 8%.

Abbreviations and Nomenclature: HKMT, histone lysine methyltransferases; PRMT, histone/protein arginine methyltransferases; H3K79, histone H3 lysine 79; MLL, mixed lineage leukemia; SAM, S-(5′-adenosyl)-L-methionine; SAH, S-(5′-adenosyl)-L-homocysteine; BOC, tert-butoxycarbonyl.

Structure based design utilizing the x-ray crystal structure of a DOT1 L/inhibitor complex revealed that N6-methyl group of the inhibitor, located favorably in a predominantly hydrophobic cavity of DOT1 L, provides a unique binding site for embodiments of the selected inhibitors provided for herein. Thus in some embodiments providing the observed high selectivity obtained by such inhibitors described herein. Further structural analysis shows that such inhibitors will also disrupt at least one H-bond and/or have steric repulsion for other histone methyltransferases binding motifs. These compounds represent novel chemical probes for biological function studies of DOT1 L in health and disease.

Human genome is packed into chromatins, which are composed of millions of repetitive units known as nucleosomes. A single nucleosome includes a fragment of DNA (˜147 bp) wound around a disc-like histone octamer consisting of two histone H2A, H2B, H3 and H4 proteins. Post-translational epigenetic modifications on several lysine and arginine residues of histones, such as methylation and acetylation, control the accessibility of the DNA, thereby regulating the expressing or silencing of a gene.¹ It has been widely recognized that, in addition to gene mutations, aberrant epigenetic modifications play an important role in the initiation of many diseases, such as cancer.²⁻⁴ Great interest has therefore been generated to study histone modifying enzymes, such as histone methyltransferases, as well as their functions in pathogenesis. Histone methyltransferases include a large family of dozens of histone lysine methyltransferases (HKMT) and histone/protein arginine methyltransferases (PRMT),^{5,6} many of which have recently been found to play critical roles in cell differentiation, gene regulation, DNA recombination and damage repair.⁷ Therefore, small molecule inhibitors of histone methyltransferases represent useful chemical probes for these biological studies as well as potential therapeutics.⁸ However, very few inhibitors of histone methyltransferases (HKMT and PRMT) have been discovered and developed.^{8,9}

Of particularly interested is human histone lysine methyltransferase DOT1 L,^{10,11} which is highly conserved from yeasts to mammals. DOT1 L is a unique HKMT in that, unlike all other HKMTs containing a SET domain (which are class V methyltransferases), it belongs to the class I methyltransferase family. In addition, DOT1 L is the only known enzyme that specifically catalyzes methylation of the histone H3-lysine79 (H3K79) residue located in the nucleosome core structure, while other methylation sites are in the unordered N-terminal tail of histone. Moreover, clinical importance of DOT1 L as well as the H3K79 methylation is that DOT1 L has been found to be necessary and sufficient for the initiation and maintenance of leukemia with MLL (mixed lineage leukemia) gene translocations.¹²⁻¹⁴ This type of leukemia accounts for ˜75% infant and ˜10% adult acute leukemia with a particularly poor prognosis.¹⁵ DOT1 L therefore represents a novel target for intervention. DOT1L inhibitors which possesses selective activity against MLL leukemia have been disclosed.¹⁶

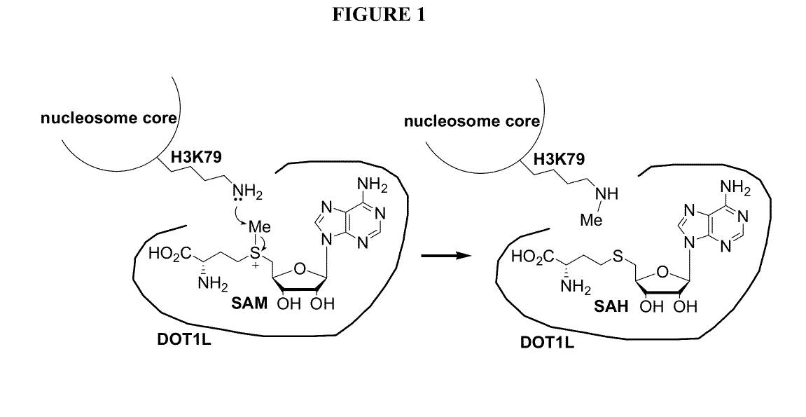

DOT1 L catalyzes an S_{N}2 reaction of the H3K79 ε-NH₂ of the substrate nucleosome with the methyl group of S-(5′-adenosyl)-L-methionine (SAM), which is the cofactor of the enzyme, as schematically illustrated in FIG. 1. One of the reaction products, S-(5′-adenosyl)-L-homocysteine (SAH) has been known to be a non-selective inhibitor of many methyltransferases, including DOT1 L.¹⁷ Herein, it was also found that it inhibits recombinant human DOT1 L (catalytic domain 1-472)¹⁰ with a Kᵢ value of 160 nM (Table 1). However, SAH cannot be used as a probe in cell biology or in vivo, since it is quickly degraded to become adenosine and homocysteine by SAH hydrolase,¹⁸ keeping cellular SAM/SAH molar ratio of ˜40:1.¹⁹ In addition, selectivity is of importance for a DOT1L inhibitor to be a useful probe, since other histone lysine and arginine methyltransferases also use SAM and histone/nucleosome as their cofactor and substrate.^{5,6}

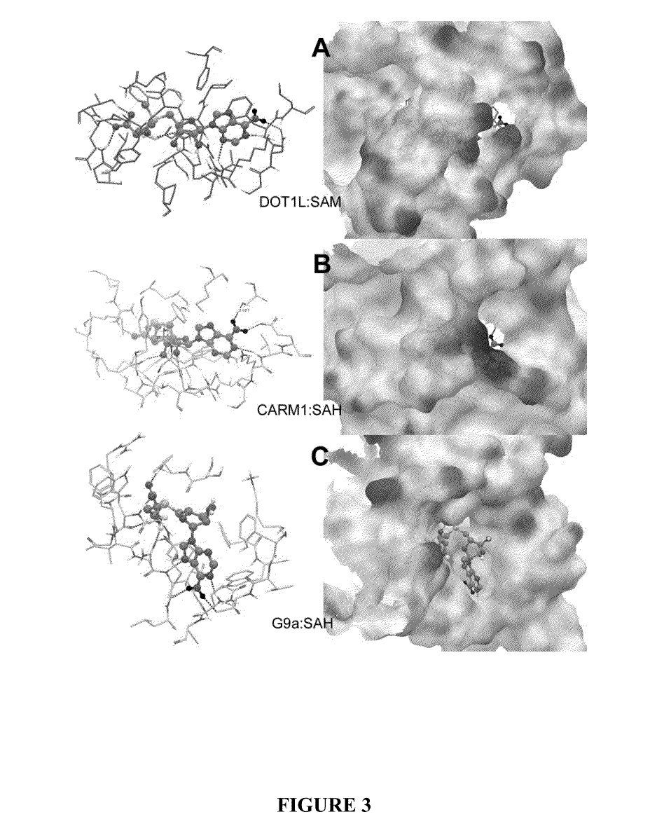

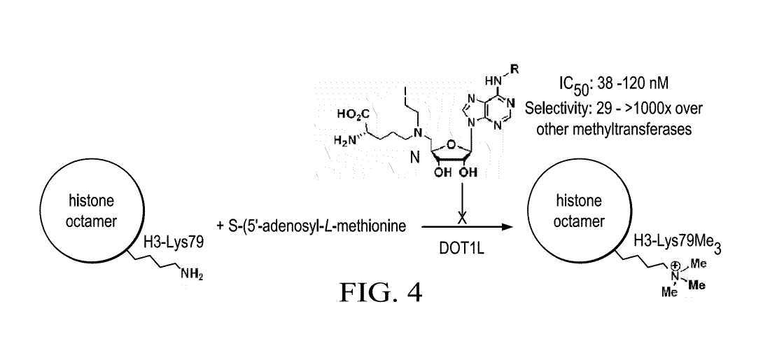

Herein, the crystal structure of the DOT1 L:SAM complex¹¹ as well as those of all other histone methyltransferases available in Protein Data Bank were analyized, and it was found that one structural feature that is unique to the binding of SAM to DOT1 L, which can be exploited to design selective DOT1 L inhibitors. As shown in Supporting Information FIG. 3a, the 6-N H₂ group of SAM forms only one H-bond with DOT1 L with a large hydrophobic cavity nearby. However, the 6-NH₂ group of bound SAM or SAH has two H-bonds with PRMTs (which also belong to class I methyltransferases), such as CARM1 (also known as PRMT4) as shown in FIG. 3b. All other HKMTs, such as G9a, are SET-domain methyltransferases having a completely different structure. The binding conformation of SAM/SAH to these enzymes is distinct from that of DOT1 L, with the 6-NH₂ group facing towards the protein and forming two H-bonds (FIG. 3c). It was thus hypothesized that N6-substituted SAH analogs, such as 1 and 2 (Chart 1),

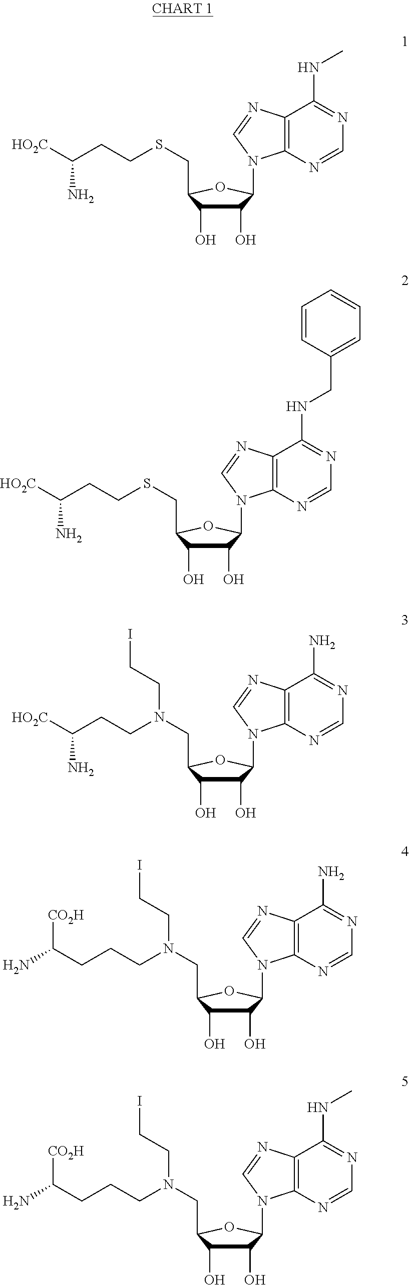

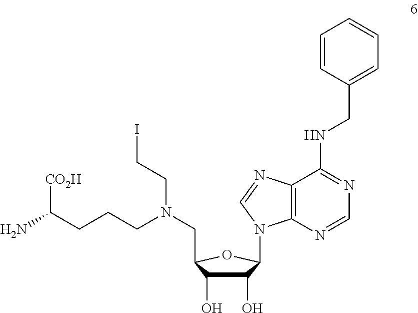

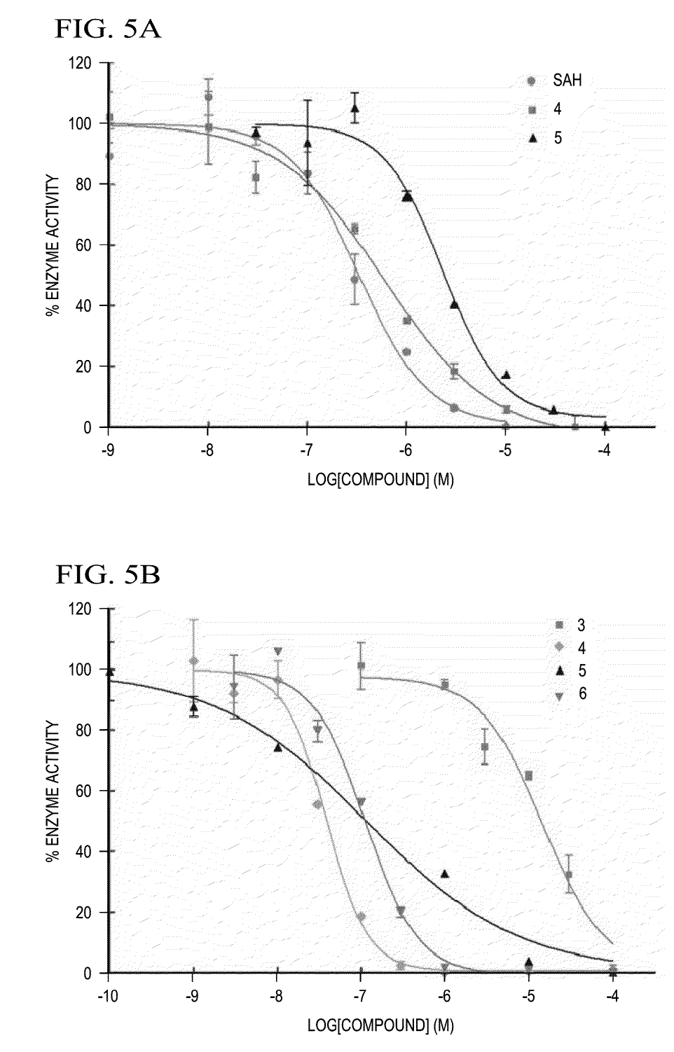

are potent and selective DOT1 L inhibitors. This turned out to be the case. Compounds 1 and 2 were synthesized from N6-substituted adenosine (Supporting Information Experimental Section). Compound 1, having only one extra —CH₃ group compared to SAH, was found to be still a potent DOT1 L inhibitor with a Kᵢ value of 290 nM (Table 1 and FIG. 5 (A-B)), but it possesses only weak or no inhibitory activities against two PRMTs (CARM1 and PRMT1) and two HKMTs (G9a and SUV39H1) with Kᵢ values of 22.7−>100 μM (Table 1). In contrast, SAH remains an inhibitor of all these enzymes with Kᵢ values of 0.4-4.9 μM. Similarly, compound 2, N6-benzyl-SAH, has good activity on DOT1 L (Kᵢ: 1.1 μM), but is very weak against CARM1 and PRMT1 (Kᵢ=18 and 21.2 μM) and inactive on G9a and SUV39H1 (Table 1).

TABLE 1
Ki or IC₅₀ (micromolar) for methyltransferease inhibitors,^{a,b}

| | DOT1L | CARM1 | PRMT1 | G9a | SUV39H1 |
|---|---|---|---|---|---|
| SAH^{a} | 0.16 | 0.40 | 0.86 | 0.57 | 4.9 |
| 1^{a} | 0.29 | >100 | 22.7 | >100 | >100 |
| 2^{a} | 1.1 | 18 | 21.2 | >100 | >100 |
| 3^{b} | 15.7 | 46.4 | 22.0 | >100 | >100 |
| 4^{b} | 0.038 | 1.1 | 2.7 | 1.8 | >100 |
| 5^{b} | 0.12 | >100 | >100 | >100 | >100 |
| 6^{b} | 0.11 | >100 | >100 | >100 | >100 |

^{a}Ki values for competitive inhibitors SAH, 1 and 2;
^{b}IC50 values for inhibitors 3-6.

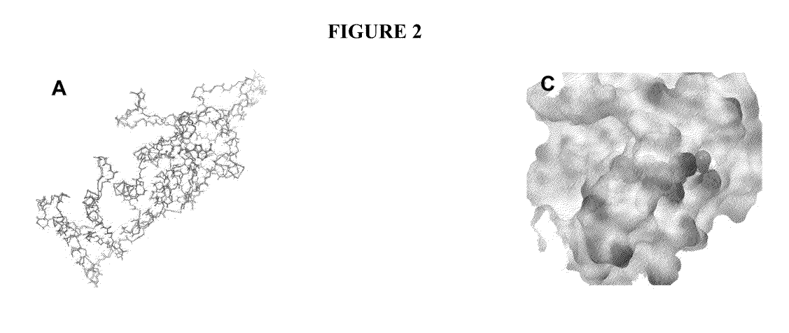

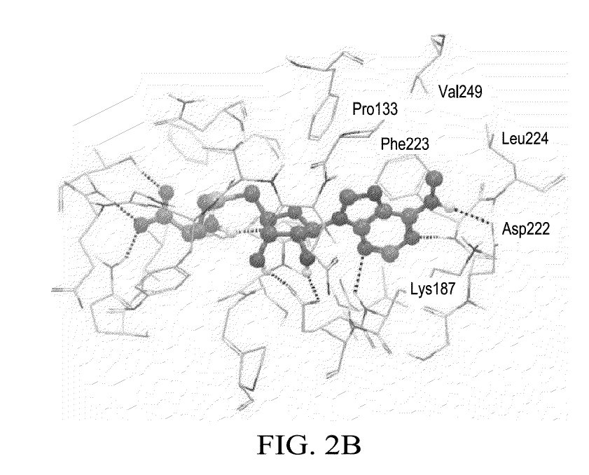

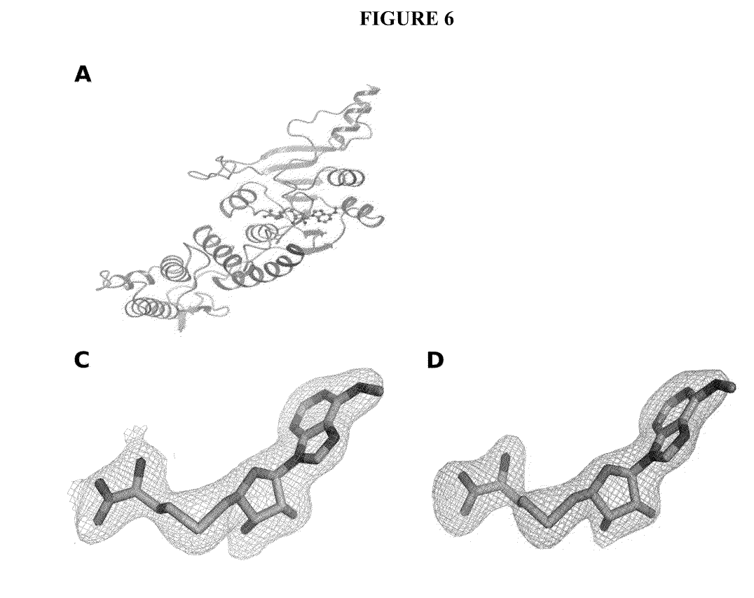

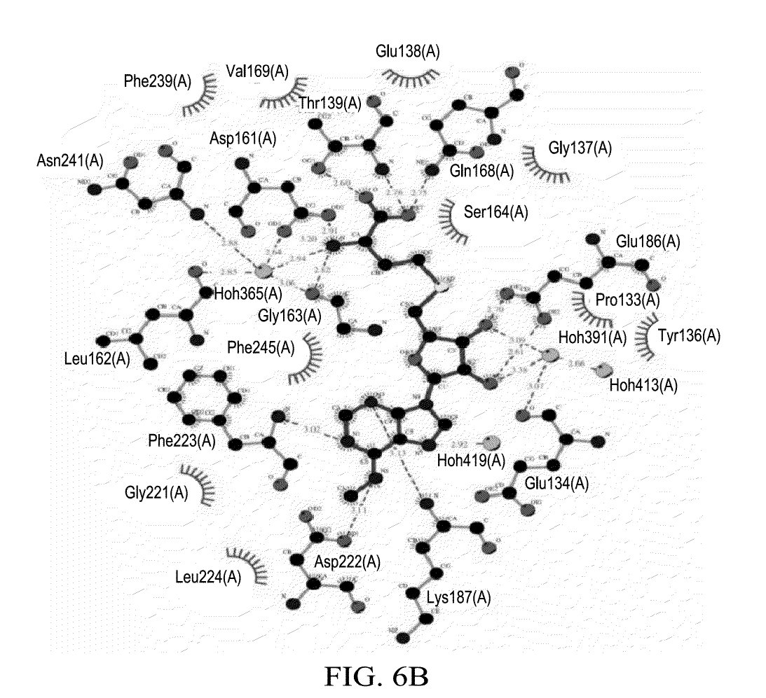

Next, x-ray crystallography was used to investigate how compound 1 binds to DOT1 L, with a particular interest in the binding site of the N6-methyl group that provides excellent selectivity. The crystal structure of the DOT1 L:1 complex was herein determined at a resolution of at 2.5 Å. Details of data processing and refinement are shown in Table 2 and the overall structure and protein-ligand interactions of the DOT1L:1 complex illustrated in FIG. 6. As shown in FIG. 2a, the protein as well as the SAH moiety of the inhibitor superimpose with those of the previously reported DOT1 L:SAM structure¹¹ with a rms (root mean square) deviation of 0.2 Å. As a result, all of the 10 H-bonds as well as other interactions between the ligand and the protein remain essentially intact (FIG. 2b), which is in agreement with the potent inhibitory activity of 1. The N6-methyl group is nicely inserted into a hydrophobic cavity, surrounded by Phe223, Leu224, Val249, Lys187 and Pro133 (FIGS. 2b,c). In addition, its orientation allows the 6-NH group to form a H-bond with Asp222 that is important to the binding of the adenine ring.

TABLE 2

| A. Data processing | |
|---|---|
| Wavelength (Å) | 1.542 |
| Space group | P6₅ |
| Unit cell dimensions | |
| a, b, c (Å) | 152.75, 152.75, 50.89 |
| α, β, γ (°) | 90.0, 90.0, 120.0 |
| Resolution (Å) | 100-2.5(2.54-2.5) |
| Unique reflections | 23040(1232) |
| Completeness (%) | 96.9(100) |
| Redundancy | 12.9(13.0) |
| R_{sym} (%) | 14.0(86.3) |
| I/σ(I)^{b} | 18.0(3.0) |
| B. Refinement | |
| Resolution (Å) | 30.55-2.5(2.54-2.5) |
| Number of reflections used in working set | 21865(1101) |
| Number of reflections for R_{free} calculation | 1154(50) |
| R_{work} (%) | 23.4(34.4) |
| R_{free} (%)^{a} | 27.4(39.2) |
| Number of all non-hydrogen atoms | 2805 |
| Number of solvent waters | 91 |
| Mean B-factor from Wilson plot (Å²) | 63.0 |
| Mean B-factor, protein atoms (Å²) | 63.7 |
| Mean B-factor, solvent atoms (Å²) | 61.9 |
| Mean B-factor, inhibitors (Å²) | 48.2 |
| Root mean square deviations from ideality | |
| Bond length (Å) | 0.007 |
| Bond Angle (°) | 1.3 |
| Dihedral (°) | 21.9 |
| Improper (°) | 0.81 |
| Ramachandran plot^{b} | |
| Residues in most favored regions | 89.8% |
| Residues in additional allowed regions | 10.2% |
| Residues in generously allowed regions | 0.0% |
| Residues in disallowed regions | 0.0% |

It is therefore clear that introducing a N6-substituent does not significantly affect the binding of SAH to DOT1 L. However, our experiments show the N6-substituted SAH analogs 1 and 2 cannot bind to other HKMTs and PRMTs strongly (Table 1), suggesting any substitution on this position will disrupt at least one H-bond and/or change the binding conformation of the adenine ring, thereby causing a considerable affinity loss. In addition, for SET-domain HKMTs, any N6-substituent will lead to intolerable steric repulsion with the protein, preventing these compounds from binding. These results show N6-substituted SAH analogs are selective inhibitors of DOT1 L and provide a structural basis for further inhibitor design and development.

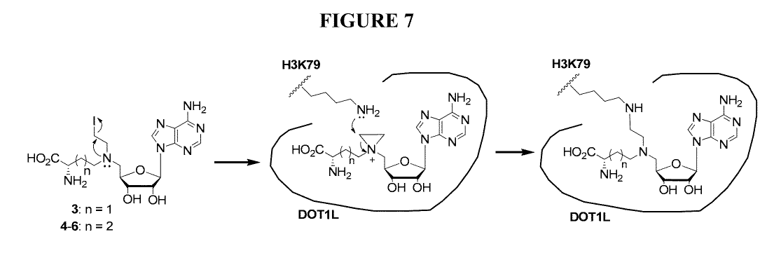

A mechanism based inhibitor design was exploited to find selective DOT1 L inhibitors with improved potency. Compound 3 (Chart 1) was initially synthesized. The rationale is that it can undergo intramolecular cyclization at neutral pH to form a reactive aziridinium intermediate,^{20,21} which may be covalently bound to the ε-NH₂ group of H3K79 (FIG. 7). Compound 3 was found to exhibit only weak enzyme inhibition against DOT1 L with an IC₅₀ value of 15.7 μM. It was reasoned that compound 4 with one more —CH₂- may be a better inhibitor, since the two C—N bonds (˜1.47 Åeach) in 3 are considerably shorter than the C—S bonds (˜1.82 Å) in SAM/SAH. The crystal structures of DOT1 L show that SAM as well as 1 bind to the protein in a fully extended conformation, suggesting the amino acid moiety of 3 might not be able to reach its optimal binding site in DOT1 L. Compound 4 has not been made before and our synthetic route is shown in Scheme 1. The 2′,3′-dihydroxyls of adenosine were selectively protected with an acetonide and the 5′-hydroxyl was converted to a —NH₂, via a Mitsunobu reaction followed by treatment with hydrazine. The product was alkylated with ethyl bromoacetate and reduced with LiAlH₄ to afford compound 7. tert-Butyl ester of L-glutamic acid was first protected with one tert-butoxycarbonyl (BOC) group and its δ-carboxyl converted to a methyl ester. It is necessary to protect the amino group with a second BOC before reduction to give aldehyde 8. Compounds 7 and 8 subjected to a reductive amination to produce compound 9, whose free hydroxyl group was converted to an iodide with PPh₃/I₂, affording, after acidic deprotection, compound 4.

Compound 4 was found to be an extremely potent inhibitor of DOT1 L with an IC₅₀ value of 38 nM (Table 1), almost quantitatively inactivating DOT1 L. Interestingly, it possesses relatively weak or no inhibitory activity on other methyltransferases with IC₅₀ values of 1.1−>100 μM, respectively, showing a high selectivity (>29-fold). Due to complicated enzyme kinetics of histone methyltransferases involving covalent binding of inhibitor 4 (or 3) to the substrate, measured IC₅₀ values for each enzyme was achieved using a minimal enzyme concentration (50-100 nM), Kₘ of SAM, as well as saturated concentration of the substrate. Under these assay conditions, the IC₅₀ values may be used to compare the relative inhibitory ability of each compound across these enzymes.

Although 4 does not have an N6-substituent, the locally more hydrophobic environment at the binding site of the putative aziridinium intermediate of 4 in DOT1 L might account for the selectivity, since it could protect the highly reactive aziridinium cation from non-specific hydrolysis. The corresponding sites in other histone methyltransferases are either exposed to the solvent (for SET domain HKMTs) or polar (for PRMTs) Compounds 5 and 6, which are N6-substituted analogs of 4, were synthesized using the general approach in Scheme 1. These two compounds also exhibit potent activity against DOT1 L with IC₅₀ values of 120 and 110 nM, respectively (Table 1). As expected, their N6-methyl and benzyl group provide excellent selectivity: 5 and 6 are essentially inactive against other methyltransferases, showing these compounds could have wide applications in probing the biological functions of DOT1 L.

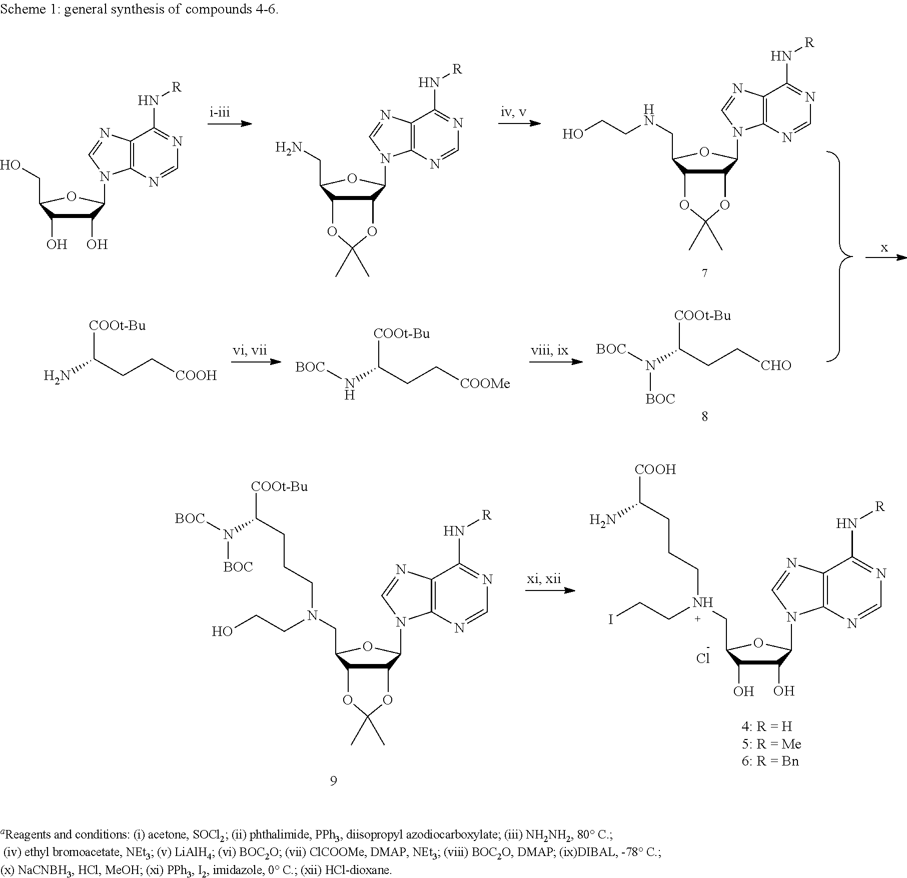

Embodiments of this disclosure thus first describe that: DOT1 L, a specific histone H3K79 methyltransferase, plays a critical role in normal cell differentiation as well as the initiation and maintenance of acute leukemia with MLL gene translocations. DOT1L inhibitors therefore represent novel chemical probes for its functional studies as well as potential therapeutics for leukemia. Secondly, structure and mechanism based design was used to synthesize and identify several potent DOT1 L inhibitors with IC₅₀ values as low as 38 nM. These compounds exhibit only weak or no inhibitory activities on four other representative histone, lysine, and arginine methyltransferases. Thirdly it was determined the crystal structure of the DOT1L:1 complex, revealing the structural basis for the excellent selectivity. The methyl group of the inhibitor is located favorably in a hydrophobic cavity of DOT1 L, while it will disrupt at least one H-bond and/or have steric repulsions for all other histone methyltransferases. This finding should provide implications for future DOT1 L inhibitor design and development.

Materials and Methods

All reagents were purchased from Alfa Aesar (Ward Hill, Mass.) or Aldrich (Milwaukee, Wis.). Compounds were characterized by ¹H NMR on a Varian (Palo Alto, Calif.) 400-MR spectrometer and the purities monitored by a Shimadzu Prominence HPLC with a Zorbax C18 or C8 column (4.6×250 mm) or using ¹H (at 400 MHz) absolute spin-count quantitative NMR analysis with imidazole as an internal standard. Identities of all new compounds were confirmed with high resolution mass spectra (HRMS) using a ThermoFisher LTQ-Orbitrap mass spectrometer.

S-(N⁶-Methyl-adenosyl)-L-homocysteine (1). It was prepared according to a literature method,² using N⁶-methyl-adenosine (562 mg, 2.0 mmol) as the starting compound, giving 1 as a white powder (270 mg, 34% overall yield). ¹H NMR (400 MHz, D₂O): δ 8.25 (s, 1 H), 8.18 (s, 1 H), 6.02 (d, J=4.4 Hz, 1 H), 4.86 (m, 1 H), 4.36 (m, 1 H), 4.28 (m, 1 H), 3.72 (m, 1 H), 3.03 (s, 3 H), 2.98-2.88 (m, 2 H), 2.63 (t, J=7.6 Hz, 2 H), 2.12-1.96 (m, 2 H).

S-(N⁶-Benzyl-adenosyl)-L-homocysteine (2). It was prepared similarly as 1 using N⁶-benzyl-adenosine (714 mg, 2.0 mmol) as the starting compound, giving 2 as a white powder (245 mg, 26% overall yield). ¹H NMR (400 MHz, D₂O): δ 8.28 (s, 1 H), 8.14 (s, 1 H), 7.33-7.15 (m, 5 H), 6.02 (d, J=4.4 Hz, 1 H), 4.80 (m, 1 H), 4.36 (m, 1 H), 4.28 (m, 1 H), 3.72 (m, 1 H), 3.30 (s, 2 H), 2.98-2.88 (m, 2 H), 2.63 (t, J=7.6 Hz, 2 H), 2.12-1.96 (m, 2 H).

N^{γ}-(5′-Adenosyl)-N^{γ}-(2-iodoethyl)-(S)-2,4-diaminobutyric acid hydrochloride (3). It was prepared according to a published procedure.^{24 1}H NMR (400 MHz, D₂O): δ 8.46 (s, 1 H), 8.45 (s, 1 H), 6.17 (d, J=4.4 Hz, 1 H), 4.86 (t, J=4.8 Hz, 1 H), 4.80 (m, 1 H), 4.51 (m, 2 H), 4.10-3.46 (m, 6 H), 3.38 (t, J=8.0 Hz, 2 H), 2.41-2.11 (m, 2 H).

N^{δ}-(5′-Adenosyl)-N^{δ}-(2-iodoethyl)-(S)-2,5-diaminopentanoic-acid hydrochloride (4). It was prepared according to Scheme 1. To a suspension of adenosine (8.03 g, 30 mmol) in 100 mL dry acetone was added trimethyl orthoformate (2.4 mL), followed by SOCl₂ (6.75 mL, 90 mmol) dropwise. After stirring overnight, the solid was filtered, dissolved in saturated NaHCO₃, and neutralized to pH ˜7. The solid was collected by filtration, washed with ether (20 mL), and dried in vacuo to give 2′,3′-isopropylidene-N⁶-methyl-adenosine, to which (3.07 g, 10 mmol) in dry THF (20 mL) were added phthalimide (1.62 g, 11 mmol) and PPh₃ (2.88 g, 11 mmol), followed by diisopropyl azodicarboxylate (DIAD, 1.08 g, 11 mmol). After 2.5 h, the white solid was filtered, washed with 20 mL of cold Et₂O. The crude Mitsunobu product and hydrazine hydrate (2.4 mL, 50 mmol) were refluxed overnight in ethanol (20 mL). After cooling, the reaction mixture was filtered and the filtrate evaporated to dryness. To the product (612 mg, 2.0 mmol) in THF (5 mL) were added Et₃N (0.9 mL, 6.4 mmol) and ethyl bromoacetate (0.28 mL, 2.5 mmol). After stirring overnight, the resulting mixture was filtered and evaporated to dryness. The residue oil thus obtained was dissolved in THF (10 mL) and cooled to −20° C., followed by addition of LiAlH₄ (166 mg, 4.4 mmol). The reaction was allowed to warm to room temperature over 2.5 h, quenched with saturated NaHCO₃, and the product was extracted with 50 mL of EtOAc, washed successively with saturated NaHCO₃, water, and saturated NaCl.

The organic layer was dried over sodium sulfate, evaporated, and purified with a flash column chromatography (silica gel, CH₂Cl₂/MeOH 95:5) to give 7 as a white solid (525 mg, 75%). Compound 7 (211 mg, 0.6 mmol) was added to a solution of aldehyde 8²⁵ (310 mg, 0.8 mmol) in anhydrous MeOH (3 mL). NaBH₃CN (67 mg, 1.05 mmol) and HCl (0.2 mL, 2.5 M in ethanol) were added to the stirring solution. The reaction mixture was stirred at room temperature overnight before being diluted with EtOAc (20 mL) and NaHCO₃. The organic layer was washed with NaHCO₃, dried over Na₂SO4, and evaporated. Purification with a column chromatography (silica gel, CH₂Cl₂/MeOH 90:10) gave 9 as a white solid (240 mg, 55%). To a solution of triphenylphosphine (77 mg, 0.30 mmol) and imidazole (20 mg, 0.30 mmol) in CH₂Cl₂ (1 mL) was added I₂ (79 mg, 0.31 mmol) at 0° C., followed by addition of 9 (140 mg, 0.19 mmol) in CH₂Cl₂ (1 mL). The reaction mixture was stirred for 2 h, diluted with ice-chilled CH₂Cl₂ (20 mL) and H₂O (5 mL). The organic layer was washed with H₂O before being cooled to 0° C., to which was added HCl in dioxane (1.5 mL, 4 N). After 1 h, the solvent was removed in vacuo and the residue was triturated with diethylether (20 mL) to give 4 as a white powder (85 mg, 75% overall yield from 9). ¹H NMR (400 MHz, D₂O): δ 8.46 (s, 1 H), 8.44 (s, 1 H), 6.15 d, J=3.6 Hz, 1 H), 4.86 (m, 1 H) 4.80 (m, 1 H), 4.57-4.46 (m, 2 H), 3.94-3.66 (m, 4 H), 3.41-3.29 (m, 4 H), 1.99-1.79 (m, 4 H). HRMS (ESI) [M+H]⁺ Calcd for C₁₇H₂₇N₇O₅I⁺: 536.1118, Found: 536.1111.

N^{δ}-[5′-(N⁶-Methyl-adenosyl)]-N^{δ}-(2-iodoethyl)-(S)-2,5-diaminopentanoic acid hydrochloride (5). It was prepared from N⁶-methyl-adenosine (562 mg, 2.0 mmol) following the above general procedure as a white powder (150 mg, 22% overall yield). ¹H NMR (400 MHz, D₂O): δ 8.43 (s, 1 H), 8.39 (s, 1 H), 6.17 (d, J=3.6 Hz, 1 H), 4.80 (m, 2 H), 4.55-4.49 (m, 2 H), 3.90 (t, J=4.8 Hz, 2 H), 3.84-3.75 (m, 2 H), 3.47-3.35 (m, 4 H), 3.21 (s, 3 H), 1.96-1.83 (m, 4 H). HRMS (ESI) [M+H]⁺ Calcd for C₁₈H₂₉N₇O₅I⁺: 550.1275, Found: 550.1255.

N^{δ}-[5′-(N⁶-Benzyl-adenosyl)]-N^{δ}-(2-iodoethyl)-(S)-2,5-diaminopentanoic acid hydrochloride (6). It was prepared from N⁶-benzyl-adenosine (803 mg, 2.25 mmol) following the above general procedure as a white powder (170 mg, 22% overall yield). ¹H NMR (400 MHz, d₆-DMSO): δ 8.44 (s, 1 H), 8.28 (s, 1 H), 7.35-7.20 (m, 5 H), 5.98 (d, J=4.0 Hz, 1 H), 4.71-4.62 (m, 2 H), 4.40 (m, 1 H), 4.23 (m, 1 H), 3.91 (m, 2 H), 3.70-3.59 (m, 2 H), 3.54-3.36 (m, 4 H), 3.20 (s, 2 H), 1.84-1.65 (m, 4 H). HRMS (ESI) [M+H]⁺ Calcd for C₂₄H₃₃N₇O₅I⁺: 626.1588, Found: 626.1576.

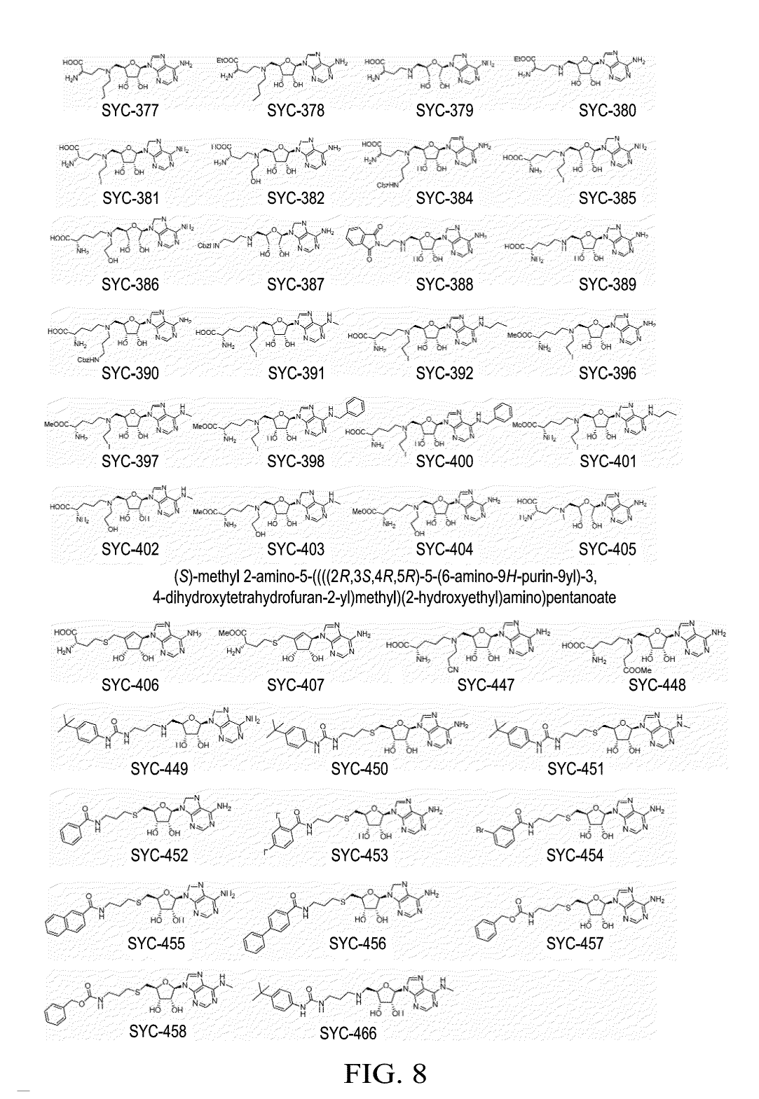

Further examples of compounds synthesized by the methods herein described are illustrated in FIG. 8 (Syc-377 through SYC-466)

Expression and purification of human DOT1 L. Human DOT1 L(1-472) was expressed and purified as described in the literature.^{26,27} In brief, BL21-CodonPlus strain (Agilent) was transformed with pGEX-KG-hDOT1L(1-472) plasmid and cultured at 37° C. in LB medium containing ampicillin (50 μg/mL) and chloramphenicol (34 μg/mL). Upon reaching an optical density of ˜1.3 at 600 nm, DOT1 L expression was induced by adding 0.2 mM isopropylthiogalactoside at 16° C. for 20 hours. Cells were harvested, lysed, centrifuged at 20,000 rpm for 20 min and the supernatant was collected and subjected to an affinity column chromatography using the glutathione sepharose resin (GE Healthcare). The GST-hDOT1L fusion protein was eluted with 10 mM of glutathione solution, and after desalting (HiTrap, GE Healthcare), the GST tag was removed by thrombin digestion overnight at 4° C. DOT1 L was purified by chromatography using a glutathione sepharose column and a Superdex 75 gel filtration column with ˜80% purity (SDS-PAGE).

hDOT1L(1-351), which is used for crystallization, was sub-cloned from pGEX-KG-hDOT1L(1-472) using 5′-TGGTGGAATTCACATGGGGGAGAAGCTGG-3′ and 5′-GACACTCGAGTCAGCTCTTGCTCTCGCGCTG-3′ as forward and reverse primers, respectively, and inserted into pGEX-KG vector. The correctness of insert was verified by sequencing. The expression and purification of hDOT1L(1-351) were similarly performed as described above.

Enzyme Inhibition Assays

PRMT1 and SUV39H1 were purchased from BPS Biosciences (San Diego, Calif.) and G9a from New England Biolabs (Ipswich, Mass.). The expression plasmid (pGEX-KG-CARM1) for human CARM1 was obtained from Dr. Qin Feng (Baylor College of Medicine). The expression and purification of CARM1 were similarly carried out as those of hDOT1 L.

Determination of Kₘ values of SAM and substrate. To determine Kₘ values of SAM, a methyltransferase (minimal amount to produce sufficient activity, ranging from 50 to 100 nM), a saturated concentration of its substrate and an increasing concentration of SAM (ranging from 0.1 to 50 μM) in 20 μL of 20 mM Tris buffer (containing 1 mM EDTA, 0.5 mM DTT and 50 μg/mL BSA, pH=8.0) were incubated at 30° C. for 10 min. The reaction was stopped by adding SAH to a final concentration of 100 μM. 15 μL of reaction mixture was then transferred to a small piece of P81 filter paper (Whatman) that binds the substrate, washed three times with 50 mM NaHCO₃, dried, and transferred into a scintillation vial containing 2 mL of scintillation cocktail. Radioactivity on the filter paper that corresponds to the amount of ³H-methyl transferred to the substrate was measured using a Beckman LS-6500 scintillation counter. Kₘ value was obtained by fitting the triplicate experimental data to Michaelis-Menten model in Prism (version 5.0, GraphPad Software, Inc., La Jolla, Calif.). The determination of Kₘ values of the substrates was done in a similar manner by varying the concentration of the substrate. Oligo-nucleosome (from chicken erythrocytes) was used as the substrate for DOT1L and CARM1, histone H4 (New England Biolabs) for PRMT1, and histone H3 peptide (1-21) (Abcam, UK) for G9a and SUV39H1.

Using this method, the Kₘ values of SAM and nucleosome for DOT1L were determined to be 0.76 and <0.05 μM, respectively, which are similar to those reported in a recent publication (Kₘ: 0.65 and 0.0086 μM).²⁸ The Kₘ values of SAM for CARM1, G9a and SUV39H1 were determined to be 1.6, 14.1 and 27.9 μM, respectively. The Kₘ value (6 μM) of SAM for PRMT1 is available from the literature.²⁹

DOT1L inhibition assay. Human DOT1L(1-472) enzyme assay was performed using 100 nM enzyme, 0.76 μM ³H-SAM (10 Ci/mM; Perkin-Elmer), 1.5 μM oligo-nucleosome in 20 μL of 20 mM Tris buffer (containing 1 mM EDTA, 0.5 mM DTT and 50 μg/mL BSA, pH=8.0). For inhibition assay, compounds with concentrations ranging from 1 nM to 100 μM were incubated with the enzyme for 10 min before adding [³H]-SAM to initiate the reaction. After 30 min at 30° C., the reaction was stopped by adding SAH to a final concentration of 100 μM. 15 μL of reaction mixture was then transferred to a small piece of P81 filter paper that binds histone H3 protein, washed three times with 50 mM NaHCO₃, dried, and transferred into a scintillation vial containing 2 mL of scintillation cocktail. Radioactivity on the filter paper was measured using a Beckman LS-6500 scintillation counter. IC₅₀ values were obtained by using a dose response curve fitting in Prism (version 5.0). FIG. 5 (A-B) shows representative dose response curves of inhibitors SAH, 1-6. The reported IC₅₀s were the mean values from at least three experiments. Kᵢ values for competitive inhibitors SAH, 1 and 2 were calculated using the Cheng-Prusoff equation Kᵢ=IC₅₀/(1+[SAM]/Kₘ).

Inhibition assays for other methyltransferases. Enzyme inhibition assays for all other histone methyltransferases were performed similarly as described above, using 50-100 nM enzyme, Kₘ of ³H-SAM, saturated concentration of the substrate (≧10×Kₘ) in 20 μL of 20 mM Tris buffer (containing 1 mM EDTA, 0.5 mM DTT and 50 μg/mL BSA, pH=8.0). Data collection and processing were carried out similarly to determine the IC₅₀ and/or Kᵢ values, using Prism.

Crystallization and structure determination. The crystallization of human DOT1 L(1-351) was carried out as described in the literature.²⁷ hDot1L(1-351) (25 mg/mL) containing 5 mM of compound 1 was crystallized under the condition of 1.25-1.7 M (NH₄)₂S₄, 0.1 M NaAc (pH 5.3). Data were collected to 2.5 A using a Rigaku FR-E+ SuperBright X-ray source at Baylor College of Medicine and processed using the program HKL2000.³⁰ The initial structure was obtained by the program Phaser³¹ using the coordinates of 1 NW3 as a target. The refinement was carried out using the program CNS,³² starting with a simulated annealing routine. The final refinement statistics were summarized in Table 2 and the coordinates were deposited into Protein Data Bank as entry 3SR4. FIGS. 2, FIGS. 3 and 6(A-D) were generated using Maestro,³³ except for FIG. 6b using Ligplot.¹³

Protein structural analysis. Protein structure analysis and visualization were performed using Maestro³³ (version 9.1) in Schrodinger suite 2010.³⁵ PDB files of the crystal structures of histone methyltransferases were imported and prepared using the module “protein preparation wizard” with default settings: water molecules (>3.0 A away from a ligand) were removed, hydrogen atoms added, ligands (substrate or inhibitor) remained in the protein structure. H-bonds were then optimized and the protein was energy-minimized using OPLS-2005 force field with all heavy atoms fixed.

Coordinates and structure factors of the DOT1 L:1 complex have been deposited in Protein Data Bank as entry 3SR4.

### REFERENCES

(1) Kouzarides, T. Cell, 2007, 128, 693.
(2) Jones, P. A.; Baylin, S. B. Cell, 2007, 128, 683.
(3) Wilson, C. B.; Rowell, E.; Sekimata, M. Nat. Rev. Immunol. 2009, 9, 91.
(4) Tsankova, N.; Renthal, W.; Kumar, A.; Nestler, E. J. Nat. Rev. Neurosci. 2007, 8, 355.
(5) Cheng, X.; Collins, R. E.; Zhang, X. Biophys. Biomol. Struct. 2005, 34, 267;
(6) Schubert, H. L.; Blumenthal, R. M.; Cheng, X. Trends Biochem. Sci. 2003, 28, 329.
(7) Bhaumik, S. R.; Smith, E.; Shilatifard, A. Nat. Struct. Mol. Biol., 2007, 14, 1008.
(8) Copeland, R. A.; Solomon, M. E.; Richon, V. M. Nat. Rev. Drug Discov. 2009, 8, 724.
(9) Cole, P. A. Nat. Chem. Biol. 2008, 4, 590.
(10) Feng, Q.; Wang, H.; Ng, H. H.; Erdjument-Bromage, H.; Tempst, P.; Struhl, K.; Zhang, Y. Curr. Biol. 2002, 12, 1052.
(11) Min, J.; Feng, Q.; Li, Z.; Zhang, Y.; Xu, R. M. Cell 2003, 112, 711.
(12) Okada, Y.; Feng, Q.; Lin, Y.; Jiang, Q.; Li, Y.; Coffield, V. M.; Su, L.; Xu, G.; Zhang, Y. Cell 2005, 121, 167.
(13) Krivtsov, A. V.; Armstrong, S. A. Nat. Rev. Cancer 2007, 7, 823.
(14) Krivtsov, A. V.; Feng, Z.; Lemieux, M. E.; Faber, J.; Vempati, S.; Sinha, A. U.; Xia, X.; Jesneck, J.; Bracken, A. P.; Silverman, L. B.; Kutok, J. L.; Kung, A. L.; Armstrong, S. A. Cancer Cell 2008, 14, 355.
(15) Hilden, J. M.; Dinndorf, P. A.; Meerbaum, S. O.; Sather, H.; Villaluna, D.; Heerema, N. A.; McGlennen, R.; Smith, F. O.; Woods, W. G.; Salzer, W. L.; Johnstone, H. S.; Dreyer, Z.; Reaman, G. H. Blood 2006, 108, 441.
(16) Daigle, S. R.; et al. Cancer Cell, 2011, 20, 53.
(17) Richon, V. M.; Johnston, D.; Sneeringer, C. J.; Jin, L.; Majer, C. R.; Elliston, K.; Jerva, L. F.; Scott, M. P.; Copeland, R. A. Chem. Biol. Drug Des. 2011, 78, 199.
(18) Borchardt, R. T.; Keller, B. T.; Patel-Thombre, U. J. Biol. Chem. 1984, 259, 4353.
(19) Chiba, P.; Wallner, C.; Kaiser, E. Biochem. Biophys. Acta 1988, 971, 38.
(20) Osborne, T.; Weller Roska, R. L.; Rajski, S. R.; Thompson, P. R. J. Am. Chem. Soc. 2008, 130, 4574.
(21) Weller, R. L.; Rajski, S. R. ChemBioChem 2006, 7, 243.
(22) Laskowski, R. A.; MacArthur, M. W.; Moss, D. S.; Thornton, J. M. J. App. Cryst. 1993, 26, 283.
(23) David, B. L.; Amal, W. Tetrahedron Lett., 2009, 50, 3939.
(24) Weller, R. L.; Rajski, S. R. ChemBioChem 2006, 7, 243.
(25) Adamczyk, M.; Johnson, D. D.; Reddy, R. E. Tetrahedron: Asymmetry 1999, 10, 775.
(26) Feng, Q.; Wang, H.; Ng, H. H.; Erdjument-Bromage, H.; Tempst, P.; Struhl, K.; Zhang, Y. Curr. Biol. 2002, 12, 1052.
(27) Min, J.; Feng, Q.; Li, Z.; Zhang, Y.; Xu, R. M. Cell 2003, 112, 711.
(28) Richon, V. M.; Johnston, D.; Sneeringer, C. J.; Jin, L.; Majer, C. R.; Elliston, K.; Jerva, L. F.; Scott, M. P.; Copeland, R. A. Chem. Biol. Drug Des. 2011, 78, 199.
(29) Rust, H. L.; Zurita-Lopez, C. I.; Clarke, S.; Thompson, P. R. Biochemistry 2011, 50, 3332.
(30) Otwinowski Z.; Minor W. In Macromolecular Crystallography, Part A, Method Enzymology, Volume 276; Carter, C. W. Jr., Sweet, R. M., Eds.; Academic Press: New York, 1997; pp 307-326.
(31) McCoy, A. J.; Grosse-Kunstleve, R. W.; Adams, P. D.; Winn, M. D.; Storoni, L. C.; Read, R. J. J. Appl. Cryst. 2007, 40, 658.
(32) Brunger, A. T.; Adams, P. D.; Clore, G. M.; DeLano, W. L.; Gros, P.; Grosse-Kunstleve, R. W.; Jiang, J. S.; Kuszewski, J.; Nilges, M.; Pannu, N. S.; Read, R. J.; Rice, L. M.; Simonson, T.; Warren, G. L. Acta Crystallogr. D Biol. Crystallogr. 1998, 54, 905.
(33) Maestro, version 9.1, Schrödinger, LLC, New York, N.Y., 2010.
(34) Wallace, A. C.; Laskowski, R. A.; Thornton, J. M. Protein Eng. 1996, 8, 127.
(35) Schrodinger suite 2010, Schrödinger, LLC, New York, N.Y., 2010.

1. A compound of formula 1 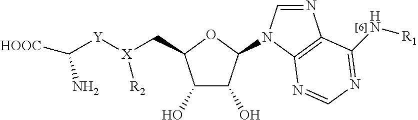
or a pharmaceutically acceptable salt or prodrug thereof, wherein
R₁ is H, methyl, or benzyl;
R₂ is 2-cyanoethyl, 2-methoxycarbonylethyl, 2-iodoethyl;
X is N or S; wherein if X═S, R₂=0; and
Y is C3 or C4, wherein said compound is selective for DOT1L Methyl Transferase.

2. A compound of formula 2 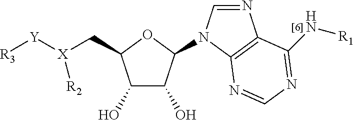
or a pharmaceutically acceptable salt or prodrug thereof, wherein
R₁ is H; alkyl; or benzyl;
R₂ is H, 2-cyanoethyl, 2-methoxycarbonylethyl, methyl, 2-iodoethyl;
ethanol; butyl; benzyl carbamate;
X is N; C; or S; wherein if XαS, R₂=0; and wherein if X═C, R₂ is also equal to R₃ or R₁, and Y is also equal to R₁, R₂ or R₃
Y is C, C₂,C₃ or C₄;
R₃ is H or selected from the following: 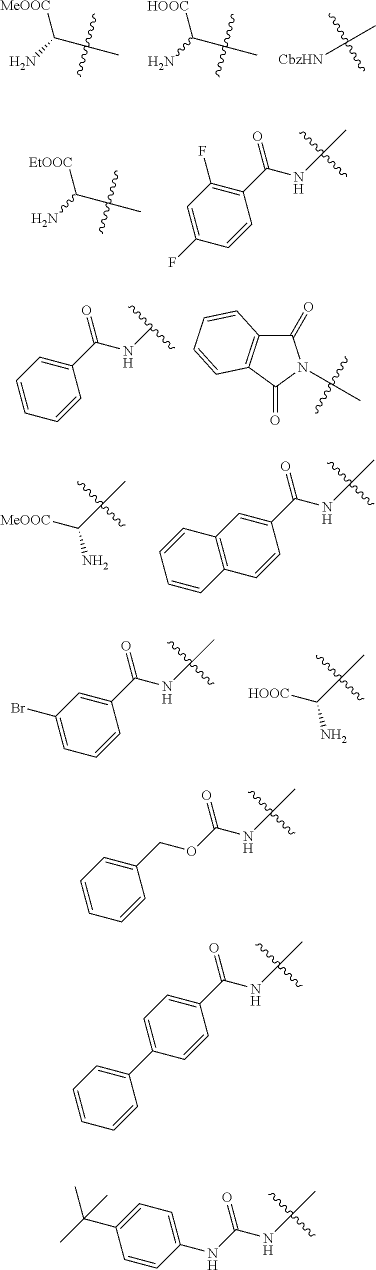
wherein said compound is selective for DOT1 L Methyl Transferase.

3. A compound of formula 3 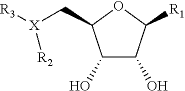
or a pharmaceutically acceptable salt or prodrug thereof, wherein
R₁ is H, or a substituted or nonsubstituted: alkyl, cycloalkyl, morpholino, aryl, biaryl, fused biaryl, benzyl; heterocycle, purine, pyrimidine, alcohol, amine, amide, aldehyde, ketone, thiol; ester, ethers, carboxylate, acyl halide, imide, amidine, nitrile, cyano, thioaldehyde, ketone, thione, thioester, thioether, hydrazines, or disulphide;
X is C, N, O or S; wherein if X═O, R₂=0; and
R₃ is H, O, or R₁;
R₂ is H, O, or R₁;
or R₃ and R₂ are cyclized together to form a substituted or nonsubstituted:
alkyl, cycloalkyl, aryl, biaryl, fused biaryl, benzyl; heterocycle, purine, pyrimidine; and
wherein said substituent may be selected from R₁, R₂, R₃, X,halide; or combinations thereof;
wherein said compound is selective for DOT1 L Methyl Transferase.

4. The compound of claim 1, wherein R1 specifically binds in the hydrophobic pocket comprising Phe 223, Leu224, Val249, Lys187 and Pro133 of DOT1 L protein, thereby selectively inhibiting DOT1 L Methyl Transferase activity.

5. The compound of claim 1, wherein the N6 hydrogen forms a hydrogen bond with Asp222 of the DOT1 L protein; thereby selectively inhibiting DOT1 L Methyl Transferase activity.

6. The compound of claim 1, wherein said compound has specificity for DOT1L and is substantially free of specificity for CARM1, PRMT1, G9a and SUV39H1 Methyl Transferases.

7. A composition comprising a compound of claim 1, a pharmaceutically acceptable salt thereof, and a pharmaceutically acceptable carrier.

8. A method of treating mixed lineage leukemia in a subject, comprising administering to the subject a therapeutically effective amount of a compound of formula 1 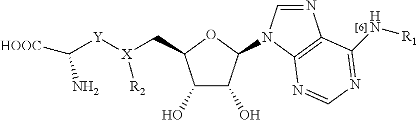
or a pharmaceutically acceptable salt or prodrug thereof, wherein
R₁ is H, methyl, or benzyl;
R₂ is 2-cyanoethyl, 2-methoxycarbonylethyl, 2-iodoethyl;
X is N or S; wherein if X═S, R₂=0; and
Y is C3 or C4, wherein said compound is selective for DOT1L Methyl Transferase.

9. The method of claim 6, wherein said compound may be administered as a prodrug; wherein said prodrug comprises replacing RCOOH or RCONH2 with an analogous alkyl ester, an aryl ester, or a heteroaryl ester.

10. A method of detecting mixed lineage leukemia comprising:
adding a diagnostically effective amount of a compound of claim 1, a pharmaceutically acceptable salt, or prodrug thereof, to an in vitro biological sample.

11. A method of detecting mixed lineage leukemia comprising:
adding a diagnostically effective amount of a compound of claim 1, or a pharmaceutically acceptable salt or prodrug thereof, to an in vitro biological sample.

12. A method of detecting mixed lineage leukemia in a subject, comprising administering to the subject a therapeutically effective amount of a compound of claim 1, or a pharmaceutically acceptable salt or prodrug thereof.

13. The method of claim 8, wherein said subject is a human.

14. The compounds of claim 1, wherein said compounds specifically inhibit methylation of histone3 lysine79 residues located in nucleosome core structure.

15. A method of treating mixed lineage leukemia in a subject, comprising administering to the subject a therapeutically effective amount of compound, wherein said compound is a structural mimic of a reaction intermediate of a compound of claim 1.
